(19) United States
(12) Patent Application Publication
(10) Pub. No.: US 2026/0256742 A1
(43) Pub. Date: Sep. 3, 2026

(54) ANDROGEN RECEPTOR ANTAGONISTS FOR THE TREATMENT OF PATIENTS WITH BIOCHEMICAL RECURRENCE OF HORMONE SENSITIVE PROSTATE CANCER

(71) Applicants: BAYER CONSUMER CARE AG, Basel (CH); ORION CORPORATION, Espoo (FI)

(72) Inventors: Lucia TRANDAFIR, Basel (CH); Patricia COLE, East Hannover, NJ (US); Iris KUSS, Berlin (DE)

(21) Appl. No.: 19/165,265

(22) PCT Filed: Mar. 15, 2024
(86) PCT No.: PCT/EP2024/056941

§ 371 Date: Sep. 15, 2025

Related U.S. Application Data
(60) Provisional application No. 63/490,619, filed on Mar. 16, 2023.

(30) Foreign Application Priority Data
Jun. 7, 2023 (EP) 23178011.5

(51) Int. Cl. A61K 31/4155; A61K 31/4166; A61K 31/4439; A61K 45/06; A61P 5/26; A61P 35/00

(52) U.S. Cl. CPC A61K 31/4155; A61K 31/4166; A61K 31/4439; A61K 45/06; A61P 5/26; A61P 35/00

(57) ABSTRACT

Described herein are methods of treating patients with biochemical recurrence of hormone-sensitive prostate cancer using a drug product comprising an androgen receptor inhibitor, such as darolutamide.

### FIELD OF THE INVENTION

Described herein are methods of treating patients with biochemical recurrence of hormone-sensitive prostate cancer using a drug product comprising for an androgen receptor inhibitor such as darolutamide.

### BACKGROUND OF THE INVENTION

Prostate cancer is the second most frequently diagnosed and fifth most aggressive neoplasm among men worldwide accounting for 13.5% (1 276 000) of the total cancer cases and 6.7% (359 000) of the total cancer deaths in males worldwide (A Barsouk, S Anand Padala, A Vakiti, et al; Epidemiology, Staging and Management of Prostate Cancer; Med. Sci. 2020, 8, 28; doi: 10.3390/medsci8030028 www.mdpi).

The course of prostate cancer from diagnosis to death is best categorized as a series of clinical stages based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate stage. Although radical prostatectomy (RP), primary definitive, radiation (RT), or a combination of both can be curative for patients with localized disease, a significant proportion (up to 50%) of these patients have recurrent disease as evidenced by a rising level of PSA with no detectable metastases, called biochemical recurrence (BCR). Generally, BCR may precede the appearance of clinical metastasis by 8 years after RP and by 7 years after primary definitive RT (Pound CR, Partin AW, Eisenberger MA, Chan DW, Pearson JD, Walsh PC. Natural history of progression after PSA elevation following radical prostatectomy. JAMA. 1999 May 5;281(17):1591-7.doi: 10.1001/jama.281.17.1591,

Zagars GK, Pollack A: Kinetics of serum prostate-specific antigen after external beam radiation for clinically localized prostate cancer. Radiother Oncol 1997; 44:213-221). The natural history of BCR and the risk of subsequent metastasis may be predicted by pre- and post-treatment clinical features (Artibani W, Porcaro AB, De Marco V, et al. Management of Biochemical Recurrence after Primary Curative Treatment for Prostate Cancer: A Review. Urol Int. 2018; 100(3):251-62). Patients with BCR are at an increased risk (24-34%) of developing metastases, which is a transition to the lethal stage of the disease (Patrikidou A, Zilli T, Baciarello G, Terisse S, Hamilou Z, Fizazi K. Should androgen deprivation therapy and other systemic treatments be used in men with prostate cancer and a rising PSA post-local treatments? Therapeutic Advances in Medical Oncology. 2021; 13. doi:10.1177/17588359211051870).

Overall, there is no treatment consensus for patients who develop BCR of hormone sensitive prostate cancer after local therapy (prostatectomy followed by adjuvant radiotherapy (ART) or salvage radiotherapy (SRT) or after primary RT), however, treatment options include androgen deprivation therapy (ADT) or local salvage therapy (S Gillessen, G Attard, T M. Beer et al; Management of Patients with Advanced Prostate Cancer: Report of the Advanced Prostate Cancer Consensus Conference 2019; European Urology 77. 2020, 508-547; Saad F, Canil C, Finelli A, Hotte SJ, Malone S, Shayegan B, et al. Controversial issues in the management of patients with advanced prostate cancer: Results from a Canadian consensus forum. Can Urol Assoc J. 2020 Apr;14(4):E137-e49). As androgen receptor (AR) signaling is a key driver of prostate cancer growth, androgen deprivation therapy (ADT) is used to suppress prostate cancer in patients with high-risk (Snaterse, G., Mies, R., van Weerden, W.M. et al. Androgen receptor mutations modulate activation by 11-oxygenated androgens and glucocorticoids. Prostate Cancer Prostatic Dis 26, 293-301 (2023)). When high-risk BCR occurs after primary or adjuvant/salvage radiotherapy, androgen deprivation therapy (ADT) may be considered a viable treatment option in patients with hormone-naïve or hormone-sensitive prostate cancer (Kim, M., Song, C., Jeong, I.G. et al. Androgen deprivation therapy during and after post-prostatectomy radiotherapy in patients with prostate cancer: a case control study. BMC Cancer 18, 271(2018 ). https://doi.org/10.1186/s12885-018-4189-9). Androgen deprivation therapy (ADT) alone is not routinely initiated in this population. When ADT is initiated, intermittent regimens of ADT are used.

How to best treat human males with BCR of hormone sensitive prostate cancer following the local treatment of prostate cancer remains an important clinical question and current evidence provides clinicians with only general guidance on how to define the risk to develop metastases considering factors such as prior therapy, PSA doubling time, factors defining high risk, time from initial therapy and patient health and preference (Artibani W, Porcaro AB, De Marco V, et al. Management of Biochemical Recurrence after Primary Curative Treatment for Prostate Cancer: A Review. Urol Int. 2018;100(3):251-62). However, there is no standard of care established for these patients (Artibani W, Porcaro AB, De Marco V, et al. Management of Biochemical Recurrence after Primary Curative Treatment for Prostate Cancer: A Review. Urol Int. 2018;100(3):251-62, Boorjian SA, Thompson RH, Tollefson MK, et al. Long term risk of clinical progression after biochemical recurrence following radical prostatectomy: the impact of time from surgery to recurrence. Eur Urol. 2011 Jun;59(6):893-9, Heinlein CA and Chang C: Androgen receptor in prostate cancer. Endoc Rev 25: 276-308, 2004, Pound CR, Partin AW, Eisenberger MA, Chan DW, Pearson JD, Walsh PC. Natural history of progression after PSA elevation following radical prostatectomy. JAMA. 1999 May 5;281(17):1591-7) Androgen depletion is the treatment option with a generally predictable outcome: decline in PSA, a period of stability in which the tumor does not proliferate, followed by rising PSA and regrowth, which may be managed by restarting androgen depletion therapy, which may be followed by a new decline in PSA, a period of stability in which the tumor does not proliferate. However, the new decline in PSA is eventually followed by PSA rising and tumor regrowth. Historically, ADT has been the standard of care for patients with metastatic prostate cancer and its use in earlier stage such as BCR is still controversial.

There is a need for a standard of care for patients with BCR hormone sensitive prostate cancer at high risk to progress to metastatic stage. In addition, patients with positive PSMA-PET are at higher-risk, and thus justify the adoption of a modern approach, including image guided radiotherapy and novel multidrug approaches for this specific patient population (Bianchi L, Ceci F, Costa F et al. The Impact of PSMA-PET on Oncologic Control in Prostate Cancer Patients Who Experienced PSA Persistence or Recurrence. Cancers. 2022 Dec 30;15(1):247. doi: 10.3390/cancers15010247).

The next-generation androgen receptor (AR) antagonist could play a role in combination with ADT in this setting. The disclosed methods are directed to these and other important needs.

### SUMMARY OF THE INVENTION

Described herein are methods of treating biochemical recurrence (BCR) of hormone sensitive prostate cancer (HSPC) comprising, consisting of, or consisting essentially of administering a safe and effective amount of at least one anti-androgen to a male human who has or is suspected to have a BCR of hormone sensitive prostate cancer. In some embodiments, the BCR of hormone sensitive prostate cancer may be a high risk BCR of hormone sensitive prostate cancer. In some embodiments, a male human with a BCR of hormone sensitive prostate cancer may have a prostate-specific antigen doubling time (PSADT) that is less than or equal to 12 months. In some embodiments, a male human with BCR of hormone sensitive prostate cancer may have Prostate Specific Membrane Antigen Positron Emission Tomography/Computed Tomography (PSMA PET/CT) lesions which are not visible on conventional imaging (bone scan and computer tomography [CT]/magnetic resonance imaging [MRI]).

In further embodiments, a male human with BCR of hormone sensitive prostate cancer may have previously received curative local therapy (radical prostatectomy which may be followed by adjuvant radiotherapy [ART] or salvage radiotherapy [RT], or primary radiotherapy). There have been no reports demonstrating that administration of the anti-androgen (darolutamide, apalutamide or enzalutamide) provides an increase in radiological progression free survival (rPFS) by Prostate Specific Membrane Antigen Positron Emission Tomography/Computed Tomography (PSMA PET/CT). However, administration of the anti-androgen darolutamide may provide a rPFS increase by PSMA PET/CT in a male human with BCR of hormone sensitive prostate cancer.

Also, there has been no reported data demonstrating that administration of (darolutamide, apalutamide or enzalutamide) provides an increase in the metastasis-free survival (MFS) as shown by conventional imaging (bone scan, CT/MRI) in patients with hormone sensitive BCR. However, administration of the anti-androgens (darolutamide, apalutamide or enzalutamide), may provide an MFS increase in a male human with BCR turned into hormone resistant prostate cancer called nmCRPC (Fizazi K, Shore N, Tammela TL, Ulys A, Vjaters E, Polyakov S, et al. Darolutamide in Nonmetastatic, Castration-Resistant Prostate Cancer. N Engl J Med. 2019 Mar 28;380(13):1235-46, Smith MR, Saad F, Chowdhury S, et al, SPARTAN Investigators. Apalutamidetreatment and metastasis-free survival in prostate cancer. N Engl J Med 2018; 378:1408-18, Hussain M, Fizazi K, Saad F, et al. Enzalutamide in men with nonmetastatic, castration-resistant prostate cancer. N Engl J Med 2018; 378:2465-74 and in patients with metastases of prostate cancer diagnosed by conventional imaging (bone scan, CT/MRI) may significantly improve overall survival and or rPFS (Chi KN, Agarwal N, Bjartell A, et al; TITAN Investigators. Apalutamide for metastatic, castration-sensitive prostate cancer. N Engl J Med. 2019;381(1):13-24. doi:10.1056/NEJMoa1903307, Armstrong AJ, Szmulewitz RZ, Petrylak DP et al (2019) ARCHES: A randomized, phase III study of androgen deprivation therapy with enzalutamide or placebo in men with metastatic hormone-sensitive prostate cancer. J Clin Oncol 37(32):2974-2986, Smith MR, Hussain M, Saad F, et al; ARASENS Trial Investigators. Darolutamide and survival in metastatic, hormone-sensitive prostate cancer. N Engl J Med. 2022;386(12):1132-1142. doi:10.1056/NEJMoa2119115). In some embodiments, administration of the anti-androgen in patients with high risk BCR may provide improved anti-tumor activity as measured by one or more of time to castration resistant prostate cancer (CRPC), time to loco-regional progression by PSMA PET/CT, PSA undetectable rates (<0.2 ng/ml) at 12 months, time to symptomatic skeletal events (SSE), overall survival (OS), time to initiation of first subsequent systemic antineoplastic therapy, time to PSA progression and PSA undetectable rates (<0.2 ng/ml) at any time.

In other embodiments, administration of a safe and effective amount of the anti-androgen results in no more than a grade 4 adverse event.

In some embodiments, the anti-androgen is a second-generation androgen receptor inhibitor. In certain embodiments, the androgen receptor inhibitor may include darolutamide, apalutamide, enzalutamide, proxalutamide or bavdegalutamide or any combination thereof. In certain embodiments, the androgen receptor inhibitor is darolutamide. In certain embodiments, the androgen receptor inhibitor is enzalutamide. In certain embodiments, the androgen receptor inhibitor is apalutamide. In further embodiments the androgen receptor inhibitor is bicalutamide, flutamide or nilutamide. In other further embodiments the androgen receptor inhibitor is ARV-766, EPI-7386, CC-94676, AC-0176, HP-518 or TAS-3681

In some embodiments, methods of treating BCR of hormone sensitive prostate cancer comprise, consist or, or consist essentially of administering a safe and effective amount of darolutamide to a male human with a BCR of hormone sensitive prostate cancer. In certain embodiments, the darolutamide is administered orally. In some embodiments, darolutamide is administered daily. In some embodiments, darolutamide is administered orally on a continuous daily dosage schedule for 24 months. In further embodiments, darolutamide is administered orally at a dose of about 1200 mg per day. In other embodiments, the darolutamide is administered orally at a dose of about 600 mg (such as by two tablets of 300 mg) taken twice daily. In some embodiments, darolutamide is present in a solid oral dosage form. In some embodiments, darolutamide is formulated as a tablet. In some embodiments, darolutamide is formulated as a soft gel. In some embodiments, darolutamide is formulated as a hard-shell capsule.

In some embodiments, the method may comprise, consist of, or consist essentially of administering a safe and effective amount of apalutamide, for example where the apalutamide is present in a solid oral dosage form. In some embodiments, apalutamide is formulated as a tablet. In some embodiments, apalutamide is formulated as a soft gel. In some embodiments, apalutamide is formulated as a hard-shell capsule.

In some embodiments, the method may comprise, consist of, or consist essentially of administering a safe and effective amount of enzalutamide, for example where the enzalutamide is present in a solid oral dosage form. In some embodiments, enzalutamide is formulated as a tablet. In some embodiments, enzalutamide is formulated as a soft gel. In some embodiments, enzalutamide is formulated as a hard-shell capsule.

According to another embodiment is a drug product which includes an antiandrogen, for instance darolutamide, and a package insert that contains instructions for improving biochemical recurrence of prostate cancer.

In certain embodiments, the invention is directed to a drug product comprising, consisting of and/or consisting essentially of darolutamide. In another aspect, in the case of darolutamide, the drug product may include a label, for example where the label for said reference listed drug includes a daily dose of 1200 mg darolutamide. In other embodiments in the case of enzalutamide, the drug product may include a label, for example where the label for the reference listed drug includes a daily dose of 160 mg enzalutamide.

In certain embodiments, the invention is directed to a method of offering for sale an approved drug product comprising, consisting of and/or consisting essentially at least one of darolutamide, apalutamide and enzalutamide. In other embodiments, the invention is directed to a method of offering for sale an approved drug product comprising, consisting of and/or consisting essentially of offering for sale of such drug product, wherein a label for a reference listed drug for such drug product includes instructions for treating non-metastatic castration resistant prostate cancer. In other embodiments, the drug product is an abbreviated new drug application (ANDA) drug product or a supplemental New Drug Application (sNDA) drug product.

These are further possible embodiments:
(A) A method of improving progression-free survival in a male human with a biochemical recurrence of prostate cancer, said method comprising administering to said male human a drug product comprising an androgen receptor inhibitor.
(B) The method of embodiment (A) wherein the progression-free survival is radiological progression-free survival by prostate-specific membrane antigen positron emission tomography.
(C) The method of embodiment (A) wherein the progression free survival is radiological progression-free survival by computed tomography.
(D) The method of embodiment (A) wherein the biochemical recurrence of prostate cancer is a high-risk biochemical recurrence of prostate cancer.
(E) The method of embodiment (A) wherein the androgen receptor inhibitor is darolutamide.
(F) The method of embodiment (A) further comprising administering an androgen deprivation therapy.
(G) The method of embodiment (F) wherein administering the androgen deprivation therapy is performed sequentially or concomitantly to the administering the drug product comprising the androgen receptor inhibitor.
(H) The method of embodiment (F) wherein the androgen deprivation therapy comprising of orchiectomy or LHRH agonists or LHRH antagonists.
(I) A method of improving radiological progression-free survival by prostate-specific membrane antigen positron emission tomography or computed tomography in a male human with a high-risk biochemical recurrence of prostate cancer, said method comprising administering to said male human an drug product comprising the androgen receptor inhibitor darolutamide in sequential or concomitant combination with androgen deprivation therapy comprising of orchiectomy or administering LHRH agonists or antagonists.
(J) A method of treating a biochemical recurrence of prostate cancer in a human male, the method comprising administering a drug product comprising an anti-androgen to the human male.
(K) The method of embodiment (J), wherein the anti-androgen is an androgen receptor inhibitor.
(L) The method of embodiment (K), wherein the androgen receptor inhibitor is a second-generation androgen receptor inhibitor selected from the group consisting of darolutamide, enzalutamide, apalutamide, and combinations of any thereof.
(M) The method of embodiment (L), wherein the second-generation androgen receptor inhibitor is administered in a daily dose ranging from about 10 mg per day to about 1500 mg per day
(N) The method of embodiment (J), wherein the androgen receptor inhibitor is darolutamide.
(O) The method of embodiment (N), wherein the darolutamide is administered in a daily dose ranging from about 300 mg per day to about 1500 mg per day.
(P) The method of embodiment (N), wherein the darolutamide is administered in a daily dose of about 1200 mg per day.
(Q) The method of embodiment (N), wherein the darolutamide is in the form of the (S, S) diastereomer, the (S, R) diastereomer, and any isomeric ratio of the (S, S) diastereomer and the (S, R) diastereomer.
(R) The method of embodiment (J), further comprising administering an androgen deprivation therapy.
(S) The method of embodiment (Q), wherein administering the androgen deprivation therapy is performed sequentially or concomitantly to the administering the drug product.
(T) Darolutamide for improving radiological progression-free survival by prostate-specific membrane antigen positron emission tomography or computed tomography in a chemically or surgically castrated male human with a high-risk biochemical recurrence of prostate cancer.

### DESCRIPTION OF THE INVENTION

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of excludes any element, step, or ingredient not specified in the claim; and (iii) ”consisting essentially of limits the scope of a claim to the specified materials or steps “and those that do not materially affect the basic and novel characteristic(s)” of the claimed invention. Embodiments described in terms of the phrase “comprising” (or its equivalents), also provide, as embodiments, those which are independently described in terms of “consisting of and “consisting essentially of.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Androgen receptor (AR) is a member of the steroid and nuclear receptor superfamily. Among this large family of proteins, only five vertebrate steroid receptors are known and include the androgen receptor, estrogen receptor alpha and beta, progesterone receptor, glucocorticoid receptor, and mineralocorticoid receptor. AR is a soluble protein that functions as an intracellular transcriptional factor. AR function is regulated by the binding of androgens, which initiates sequential conformational changes of the receptor that affect receptor-protein interactions and receptor-DNA interactions. AR is mainly expressed in androgen target tissues, such as the prostate, skeletal muscle, liver, and central nervous system (CNS), with the highest expression level observed in the prostate, adrenal gland, and epididymis. AR can be activated by the binding of endogenous androgens, including testosterone and 5a-dihydrotestosterone (5a-DHT).

The androgen receptor (AR), located on chromosome Xq 11-12, is a 110 kD nuclear receptor that, upon activation by androgens, mediates transcription of target genes that modulate growth and differentiation of prostate epithelial cells. Similar to the other steroid receptors, unbound AR is mainly located in the cytoplasm and associated with a complex of heat shock proteins (HSPs) through interactions with the ligand-binding domain. Upon agonist binding, AR goes through a series of conformational changes: the heat shock proteins dissociate from AR, and the transformed AR undergoes dimerization, phosphorylation, and translocation to the nucleus, which is mediated by the nuclear localization signal. Translocated receptor then binds to the androgen response element (ARE), which is characterized by the six-nucleotide half-site consensus sequence 5′-TGTTCT-3′ spaced by three random nucleotides and is located in the promoter or enhancer region of AR gene targets. Recruitment of other transcription co-regulators (including co-activators and co-repressors) and transcriptional machinery further ensures the transactivation of AR-regulated gene expression. All of these processes are initiated by the ligand-induced conformational changes in the ligand-binding domain.

Prostate cancer is the second most common cause of cancer death in men in the US, and approximately one in every six American men will be diagnosed with the disease during his lifetime. Treatment is aimed at eradicating the tumor, however, after primary therapy, up to 50% of patients with prostate cancer develop biochemical recurrence (BCR) defined as rising PSA with no evidence of metastatic disease by conventional imaging (CI) (Lin et al. 2019). Patients with BCR are at an increased risk of developing metastasis and despite the recent progress, the treatment of metastatic prostate cancer is still palliative, (Boorjian et al. 2011, Heinlein and Chang 2004, Jackson et al. 2018, Pound et al. 1999). Given that prostate cancer cells depend on the androgen receptor (AR) for their proliferation and survival, some men with BCR are treated with agents that block production of testosterone (e.g., GnRH agonists) called androgen deprivation therapy (ADT), alone or in combination with anti-androgens (e.g., bicalutamide), which antagonize the effect of any residual testosterone on AR. The approach is effective as evidenced by a drop in PSA and regression of visible tumor (if present) in some patients; however, this is generally followed by regrowth which can be retreated with ADT. This may be followed by a new PSA decrease and tumor regression, which is eventually followed by regrowth as a castration resistant prostate cancer (CRPC) to which most patients eventually succumb. Recent studies on the molecular basis of CRPC have demonstrated that CRPC continues to depend on AR signaling and that a key mechanism of acquired resistance is an elevated level of AR protein (C.D. Chen et al., Nat. Med, 2004, 10, 33-39). AR targeting agents with activity in castration sensitive and castration resistant prostate cancer have great promise in treating this lethal disease.

The course of prostate cancer from diagnosis to death is best categorized as a series of clinical states based on the extent of disease, hormonal status, and absence or presence of detectable metastases by conventional imaging (CT/MRI and bone scans): localized disease, rising levels of PSA after radiation therapy or surgery with no detectable metastases by conventional imaging, and clinical metastases in the non-castrate or castrate state. Although surgery, radiation, or a combination of both can be curative for patients with localized disease, a significant proportion of these patients have recurrent disease as evidenced by a rising level of PSA, which can lead to the development of metastases, especially in the high-risk group—a transition to the lethal phenotype of the disease.

Androgen depletion is the treatment option with a generally predictable outcome: decline in PSA, a period of stability in which the tumor does not proliferate, followed by rising PSA and regrowth. Anti-androgens are useful for the treatment of prostate cancer during its early stages. However, prostate cancer often advances to a ‘hormone-refractory’ state in which the disease progresses in the presence of continued androgen ablation or anti-androgen therapy. Instances of anti-androgen withdrawal syndrome have also been reported after prolonged treatment with antiandrogens. Antiandrogen withdrawal syndrome is commonly observed clinically and is defined in terms of the tumor regression or symptomatic relief observed upon cessation of anti-androgen therapy. AR mutations that result in receptor promiscuity and the ability of these anti-androgens to exhibit agonist activity might at least partially account for this phenomenon. For example, hydroxyflutamide and bicalutamide act as AR agonists in T878A and W742L/W742C AR mutants (H.L. Liu et al., Int. J. Mol. Sci., 2017, 18:1823; T. Hara et al., Cancer Res., 2003, 63:149-153), respectively, and enzalutamide and apalutamide act as AR agonist in F877L AR mutant (J.D. Joseph, Cancer Discov., 2013, 3:1020-1029).

In the setting of prostate cancer cells that were rendered castration resistant via overexpression of AR, it has been demonstrated that certain anti-androgen compounds, such as bicalutamide, have a mixed antagonist/agonist profile (C. Tran et al., Science, 2009,324:787-90). This agonist activity helps to explain a clinical observation, called the anti-androgen withdrawal syndrome, whereby about 30% of men who progress on AR antagonists experience a decrease in serum PSA when therapy is discontinued (H.I. Scher et al., J. Clin. Oncol., 1993. 11:1566-72).

### Prostate Cancer Stages

In the early stages of prostate cancer, the cancer is localized to the prostate. In these early stages, treatment typically involves either surgical removal of the prostate or radiation therapy to the prostate or observation only with no active intervention therapy in some patients. In the early stages where the prostate cancer is localized and requires intervention, surgery or radiation therapy are curative by eradicating the cancerous cells. Up to 50% of the time these procedures fail, and the prostate cancer continues to progress, as typically evidenced by a rising PSA level. Men whose prostate cancer has progressed following these early treatment strategies are said to have advanced or recurrent prostate cancer.

Because prostate cancer cells depend on the androgen receptor (AR) for their proliferation and survival, men with advanced prostate cancer are treated with agents that block the production of testosterone (e.g., GnRH agonists), alone or in combination with anti-androgens (e.g., bicalutamide), which antagonize the effect of any residual testosterone on AR. These treatments reduce serum testosterone to castrate levels, which generally slows disease progression for a period of time. The approach is effective as evidenced by a drop in PSA and the regression of visible tumors in some patients.

Eventually, the regrowth is no more followed by PSA drop defined as castration-resistant prostate cancer (CRPC), to which most patients eventually succumb.

In some embodiments, prior to treatment with a second-generation anti-androgen as described herein, men with BCR of hormone sensitive prostate cancer are characterized as having the following:
1. Histologically or cytologically confirmed adenocarcinoma of the prostate without small cell, ductal carcinoma component or with <50% neuroendocrine differentiation with high risk defined as Prostate-specific antigen doubling time (PSADT) <12 months and PSA ≥0.2 ng/ml after post radical prostatectomy which may be followed by ART or SRT, or PSA ≥2 ng/ml above the nadir after primary radiotherapy.
2. Hormone sensitive prostate cancer demonstrated by serum testosterone ≥150 ng/dl (5.2 nmol/L).
3. Presence of at least one positive lesion of prostate cancer identified by prostate-specific membrane antigen positron emission tomography/computed tomography (PSMA PET/CT) AND.
4. Absence of metastasis by bone scan, CT or MRI scans

### BCR Differs from Other Prostate Cancers

Overall, most patients with prostate cancer are diagnosed at a localized or locoregional stage. The critical moment is the failure of primary treatment (surgery and or radiotherapy).

The prostate-specific antigen (PSA) increase without macroscopically detectable disease is defined as biochemical recurrence (BCR). The definition of BCR depends on the type of prior definitive therapy. In patients who have undergone radical prostatectomie (RP), the European Association of Urology (EAU) 2020 guidelines propose that a rising serum PSA level should be considered a BCR.

Androgen receptor (AR) signaling is the key driver of prostate cancer growth. Androgen deprivation therapy (ADT) is used to suppress prostate cancer cells in patients with high-risk BCR (Snaterse, G., Mies, R., van Weerden, W.M. et al. Androgen receptor mutations modulate activation by 11-oxygenated androgens and glucocorticoids. Prostate Cancer Prostatic Dis 26, 293-301 (2023). https://doi.org/10.1038/s41391-022-00491-z). Therefore, ADT was used for decades as treatment of hormone-naïve BCR. Regardless the regimen used (intermittent or continued), patients eventually will develop prostate cancer resistance to ADT, defined as nmCRPC if the resistance is defined only by PSA increase, or mCRPC if the treatment resistance is defined by the presence of metastases identified by conventional imaging (bone scan, CT/MRI).

Androgen receptor inhibitors including enzalutamide, apalutamide and darolutamide in combination with ADT significantly increased metastatic free survival (MFS) and overall survival (OS) in patients with nmCRPC.

The PSMA PET radiotracers, 68Ga-PSMA-11 and 18F-DCFPyL have been approved by FDA in 2020 and 2021 for patients with newly diagnosed high-risk disease or patients with biochemical recurrent disease (BCR) respectively. The high sensitivity of PSMA PET/CT will permit to detected prostate cancer lesions in patients with BCR and this patient population will be a challenge for clinicians, because currently, there is no clear standards of care for these patients. Patients who develop BCR after definitive surgery or radiation and who have exhausted their salvage options, will represent even a greater challenge. These patients were considered to have BCR because they were ineligible for all the trials in mHSPC (Davis ID, Martin AJ, Stockler MR, et al: Enzalutamide with standard first-line therapy in metastatic prostate cancer. N Engl J Med 381:121-131, 2019 17; Fizazi K, Tran N, Fein L, et al: Abiraterone plus prednisone in metastatic, castration-sensitive prostate cancer. N Engl J Med 377:352-360, 2017; Sweeney CJ, Chen YH, Carducci M, et al: Chemohormonal therapy in metastatic hormone-sensitive prostate cancer. N Engl J Med 373:737-746, 2015; Smith MR, MHussain M, Saad F, et al: Darolutamide and survival in metastatic, hormone-sensitive prostate cancer. N Engl J Med 386:1132-1142, 2022)

Although some clinicians may consider the extrapolation of PSMA PET/CT lesions to mHSPC, these patient population with less advanced disease might not have the same benefit (Madan RA, Mena E, Lindenberg L, Choyke PL. With New Technology Comes Great Responsibility: Prostate-Specific Membrane Antigen Imaging in Recurrent Prostate Cancer. J Clin Oncol. 2022 Sep 10; 40(26):3015-3019. doi: 10.1200/JCO.22.00493.). Most patients in mHSPC trials had de novo disease, aggressive enough to evolve despite PSA screening. Biochemical recurrence may evolve many years after initial treatment (surgery and/or radiation) and requires different therapeutic approach upon detection on PSMA PET imaging. Treatments that have established benefit in quite a different clinical population such as mHSPC, could not be appropriate in patients with BCR and PSMA PET/CT lesions.

An important question that is being evaluated in ARASTEP is, whether systemic therapy with imaging (PSMA PET/CT)-guided therapy may achieve durable response.

### Anti-Androgens

As used herein, the term “anti-androgen” refers to a group of hormone receptor antagonist compounds that are capable of preventing or inhibiting the biologic effects of androgens on normally responsive tissues in the body. In some embodiments, an anti-androgen is a small molecule. In some embodiments, an anti-androgen is an AR antagonist. In some embodiments, an anti-androgen is an AR full antagonist. In some embodiments, an anti-androgen is a first-generation anti-androgen. In some embodiments, an anti-androgen is a second-generation anti-androgen.

As used herein, the term “AR antagonist” or “AR inhibitor” are used interchangeably and refer to an agent that inhibits or reduces at least one activity of an AR polypeptide. Exemplary AR activities include, but are not limited to, co-activator binding, DNA binding, ligand binding, or nuclear translocation.

As used herein, a “full antagonist” refers to an antagonist, which, at an effective concentration, essentially completely inhibits an activity of an AR polypeptide. As used herein, a “partial antagonist” refers an antagonist that is capable of partially inhibiting an activity of an AR polypeptide, but that, even at a highest concentration is not a full antagonist. By ‘essentially completely’ is meant at least about 80%, at least about 90%, at least about 95%, at least about 96%, at least about 97%, at least about 98% at least about 99%, or greater inhibition of the activity of an AR polypeptide.

As used herein, the term “second-generation anti-androgen” refers to an agent that exhibits full antagonist activity against a wild-type AR polypeptide. Second-generation anti-androgens differ from first-generation anti-androgens in that second-generation anti-androgens act as full antagonists in cells expressing elevated levels of AR, such as for example, in castration resistant prostate cancers (CRPC). Exemplary second-generation anti-androgens include darolutamide, apalutamide, enzalutamide, proxalutamide or bavdegalutamide, bicalutamide, flutamide or nilutamide, or ARV-766, EPI-7386, CC-94676, AC-0176, HP-518 or TAS-3681.

In some embodiments, an anti-androgen contemplated in the methods described herein inhibits AR nuclear translocation, such as darolutamide, DNA binding to androgen response elements, and coactivator recruitment. In some embodiments, an anti-androgen contemplated in the methods described herein exhibits no agonist activity in AR-overexpressing prostate cancer cells.

Darolutamide is a second-generation anti-androgen that binds directly to the ligand-binding domain of the AR, impairing nuclear translocation, AR binding to DNA and AR target gene modulation, thereby inhibiting tumor growth. Darolutamide binds to the AR with greater affinity than bicalutamide, and induces partial or complete tumor regression in non-castrate hormone-sensitive and bicalutamide-resistant human prostate cancer xenograft models (A. Moilanen et al., Sci. Rep., 2016, 5:12007; T. Sugawara et al., Int. J. Cancer, 2019, 14:1382-1394). Darolutamide lacks the partial agonist activity seen with bicalutamide in the context of AR overexpression.

Darolutamide, BAY1841788 or ODM-201, is an AR antagonist consisting of two diastereomers—ORM-16497 and ORM-16555 (K. Fizazi et al., Lancet Oncol., 2014, 15:975-985; T. Sugawara et al., Int. J. Cancer, 2019, 14:1394). It has activity against known AR mutants that confer resistance to other second-generation antiandrogens (H. Borgmann et al., 2018, Eur. Urol., 2018, 73:4-8; T. Sugawara et al., Int. J. Cancer, 2019, 14:1394). Darolutamide binds to the AR with high affinity, and impairs subsequent androgen-induced nuclear translocation of AR and transcription of AR gene target (T. Sugawara et al., Int. J. Cancer, 2019, 14:1394; S.J. Baumgart et al., Mol. Oncol., 2021, 14:2022-2039).

In one aspect described herein are methods of BCR hormone sensitive prostate cancer comprising, consisting of, or consisting essentially of administering a safe and effective amount of an anti-androgen to a male human with a BCR of hormone sensitive prostate cancer. In another aspect described herein are methods of treating a male human having BCR of hormone sensitive prostate cancer comprising, consisting of, or consisting essentially of administering a safe and effective amount of an anti-androgen to a male human with a BCR of hormone sensitive prostate cancer. In the following disclosure, “methods of treating BCR of hormone sensitive prostate cancer,” may alternatively be recited as “methods of treating a male human having BCR of hormone sensitive prostate cancer.” For the sake of brevity, each possible alternative is not parsed out.

### Further Definitions

The term “cancer” as used herein refers to an abnormal growth of cells which tend to proliferate in an uncontrolled way and, in some cases, to metastasize (spread).

The term “prostate cancer” as used herein refers to histologically or cytologically confirmed adenocarcinoma of the prostate.

The term “androgen-deprivation therapy (ADT)” refers to the reduction of androgen levels in a prostate cancer patient to castrated levels of testosterone (<50 ng/dL). Such treatments can include orchiectomy or the use of gonadotropin-releasing hormone “GnRH” agonists or antagonists. ADT includes surgical castration (orchiectomy) and/or the administration of luteinizing hormone-releasing hormone (“LHRH”) agonists or antagonists to a human (chemical castration). Examples of LHRH agonists include goserelin acetate, histrelin acetate, leuprolide acetate, and triptorelin palmoate. Physicians can prescribe LHRH agonists in accordance with instructions, recommendations and practices. This may include about 0.01 mg to about 20 mg of goserelin over a period of about 28 days to about 3 months, preferably about 3.6 mg to about 10.8 mg of goserelin over a period of about 28 days to about 3 months; about 0.01 mg to about 200 mg of leuprolide over a period of about 3 days to about 12 months, preferably about 3.6 mg of leuprolide over a period of about 3 days to about 12 months; or about 0.01 mg to about 20 mg of triptorelin over a period of about 1 month, preferably about 3.75 mg of triptorelin over a period of 1 month. About 50 mg of histrelin acetate over a period of 12 months of histrelin acetate or about 50μg per day of histrelin acetate.

The term “locally advanced prostate cancer” refers to prostate cancer where all actively cancerous cells appear to be confined to the prostate and the associated organs or neighbor organs (e.g., seminal vesicle, bladder neck, and rectal wall).

The term “high-risk localized prostate cancer” refers to locally advanced prostate cancer that has a probability of developing metastases or recurrent disease after primary therapy with curative intent. In some embodiments, high risk for development of metastases is defined as prostate specific antigen doubling time (PSADT)<20 months, <19 months, <18 months, <17 months, <16 months, <15 months, <14 months, <13 months, <12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having a high Gleason score or bulky tumor.

The term “castration-sensitive prostate cancer” or hormone-sensitive prostate cancer refers to cancer that is responsive to androgen-deprivation therapy (ADT) either as localized disease, biochemical relapse or in the metastatic setting.

The term “metastatic castration-sensitive prostate cancer” or metastatic hormone-sensitive prostate cancer refers to cancer that has spread (metastasized) to other areas of the body, e.g., the bone, lymph nodes or other parts of the body in a male, and that is responsive to androgen-deprivation therapy (ADT).

The term “non-metastatic castration-sensitive prostate cancer” or non-metastatic hormone-sensitive prostate cancer refers to cancer that has not spread (metastasized) in a male, and that is responsive to androgen-deprivation therapy (ADT). In some embodiments, non-metastatic castration-sensitive prostate cancer is assessed with bone scan and computed tomography (CT) or magnetic resonance imaging (MRI) scans.

The term “CRPC” as used herein refers to castration-resistant prostate cancer. CRPC is prostate cancer that continues to grow despite the suppression of male hormones that fuel the growth of prostate cancer cells.

The term “metastatic castration-resistant prostate cancer” refers to castration-resistant prostate cancer that has metastasized to other parts of the human body.

The term “NM-CRPC” as used herein refers to non-metastatic castration-resistant prostate cancer. In some embodiments, NM-CRPC is assessed with bone scan and computed tomography (CT) or magnetic resonance imaging (MRI) scans.

In some embodiments, the non-metastatic castration-resistant prostate cancer is a high-risk non-metastatic castration-resistant prostate cancer. The term “high risk NM-CRPC” refers to probability of a man with NM-CRPC developing metastases. In some embodiments, high risk for development of metastases is defined as prostate specific antigen doubling time (PSADT)<20 months, <19 months, <18 months, <17 months, <16 months, <15 months, <14 months, <13 months, <12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having local-regional recurrence (e.g. primary tumor bed, bladder neck, anastomotic area, pelvic lymph nodes).

The terms “co-administration” or the like, as used herein, encompass administration of the selected therapeutic agents to a single patient, and are intended to include treatment regimens in which the agents are administered by the same or different route of administration and/or at the same or different time.

The term “pharmaceutical combination” as used herein, means a product that results from the mixing or combining of more than one active ingredient and includes both fixed and non-fixed combinations of the active ingredients. The term “fixed combination” means that the active ingredients, e.g., the anti-androgen and a co-agent, are both administered to a patient simultaneously in the form of a single unit or single dosage form. The term “non-fixed combination” means that the active ingredients, e.g., the anti-androgen and a co-agent, are administered to a patient as separate units or separate dosage forms, either simultaneously, concurrently or sequentially with no specific intervening time limits, wherein such administration provides safe and effective levels of the two active ingredients in the body of the human male. The latter also applies to cocktail therapy, e.g., the administration of three or more active ingredients.

The term “PSMA_PET” refers to Prostate-specific membrane antigen Positron Emission Tomography and is a technique that uses a tracer based on the presence of prostate-specific membrane antigen (PSMA) on prostate cancer cells, and allows for a visual assessment of ligand binding to the androgen receptor in a patient. It may be used to evaluate pharmacodynamics of an androgen receptor directed therapy.

The term “continuous daily dosing schedule” refers to the administration of a particular therapeutic agent without any drug holidays from the particular therapeutic agent. In some embodiments, a continuous daily dosing schedule of a particular therapeutic agent comprises administration of a particular therapeutic agent every day at roughly the same time each day.

The terms “treat” and “treatment” refer to the treatment of a patient afflicted with a pathological condition and refers to an effect that alleviates the condition by killing the cancerous cells, but also to an effect that results in the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “radiological progression-free survival by PSMA PET/CT” or “rPFS” is based on PSMA PET/CT and is defined as is defined as the time from randomization to first documentation of at least one new distant metastatic lesion by PSMA PET/CT or death due to any cause, whichever occurs first. A distant metastatic lesion is one occurring in bone, viscera or in lymph nodes above the common iliac artery (CIA) bifurcation/distal to external iliac artery.

The term “metastasis-free survival” or “MFS” refers to the percentage of subjects in a study who have survived without cancer spread for a defined period of time or death. MFS is usually reported as time from the beginning of enrollment, randomization or treatment in the study. MFS is reported for an individual or a study population. In the context of treatment of CRPC with an anti-androgen, an increase in the metastasis-free survival is the additional time that is observed without cancer having spread or death, whichever occurs first, as compared to treatment with placebo. In some embodiments, the increase in the metastasis-free survival is about 1 month, about 2 months, about 3 months, about 4 months, about 5 months, about 6 months, about 7 months, about 8 months, about 10 months, about 11 months, about 12 months, about 13 months, about 14 months, about 15 months, about 16 months, about 17 months, about 18 months, about 19 months, about 20 months, or greater than 20 months. In some embodiments, administration of a safe and effective amount of an anti-androgen provides an increase in the metastasis-free survival of a male human, optionally wherein the increase in the metastasis-free survival is relative to the mean survival rate of a population of male humans with the non-metastatic castration-resistant prostate cancer, said population having been treated with a placebo. In some embodiments, metastasis-free survival refers to the time from randomization to the time of first evidence of BICR-confirmed bone or soft tissue distant metastasis or death due to any cause, whichever occurs first.

The term “time to CRPC” is defined as the time from randomization to first castration-resistant event defined as the time to PSA progression with serum testosterone being at castrate level <0.50 ng/ml, or time to radiological progression by CI whichever occurs first.

The term “time to first symptomatic skeletal event (SSE)” is defined as the time from randomization to time of first occurrence of SSE. An SSE is defined as the occurrence of one of the following types of events: occurrence of spinal cord compression, pathologic bone fracture, tumor-related orthopedic surgical intervention or external beam radiation therapy (EBRT) to relieve skeletal symptoms

The term “time to loco-regional progression by PSMA PET/CT” is defined as the time from randomization to time of first documentation of loco-regional progression by PSMA PET/CT or death due to any cause, whichever occurs first.

The term “PSA undetectable rate at 12 months” is defined as the percentage of participants with detectable PSA values (20.2 ng/ml) at baseline, which become undetectable (<0.2 ng/ml) at 12 months.

The term “time to deterioration in the FACT-P total score” as time to QoL deterioration is defined as the time from randomization to a 10-point reduction of FACT-P total score.

The term “overall survival” is defined as the time from randomization to the date of death due to any cause. Survival data for subjects who are alive at the time of the analysis was to be censored on the last known date that they were alive. In addition, for subjects with no post-baseline information survival, data was to be censored on the date of randomization; for subjects who are lost to follow-up or who withdraw consent, data is censored on the last known date that they were alive. In some embodiments, administration of a safe and effective amount of an anti-androgen provides improved anti-tumor activity as measured by overall survival.

The term “time to initiation of first subsequent systemic antineoplastic therapy” is defined as the time from randomization to documentation of first subsequent systemic antineoplastic therapy being administered to the subject (e.g., survival follow-up CRF). Time to initiation of first subsequent systemic antineoplastic therapy for subjects who do not start subsequent systemic antineoplastic therapy is censored on the date of last contact. In some embodiments, administration of a safe and effective amount of an anti-androgen provides improved anti-tumor activity as measured by time to cytotoxic chemotherapy.

The term “placebo” as used herein means administration of a pharmaceutical composition that does not include a second-generation anti-androgen. In the context of treatment of CRPC, men that are administered an anti-androgen or placebo will need to continue to maintain castrated levels of testosterone by either co-administration of a GnRH agonist/antagonist or orchiectomy.

The term ‘randomization’ as it refers to a clinical trial refers to the time when the patient is confirmed eligible for the clinical trial and gets assigned to a treatment arm.

The terms “kit” and “article of manufacture” are used as synonyms.

The term “subject” and “patient” and “human” are used interchangeably.

The term, “drug product” is a product that contains an active pharmaceutical ingredient.

The terms “sale” or “selling” means transferring a drug product, e.g., a pharmaceutical composition or an oral dosage form, from a seller to a buyer.

The term “offering for sale” means the proposal of a sale by a seller to a buyer for a drug product, e.g., a pharmaceutical composition and an oral dosage form.

### Routes of Administration and Pharmaceutical Compositions

Therapeutic agents described herein are administered in any suitable manner or suitable formulation. Suitable routes of administration of the therapeutic agents include, but are not limited to, oral and parenteral (e.g., intravenous, subcutaneous, intramuscular). All formulations are in dosages suitable for administration to a human. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

The term “safe and effective amount” refers to an amount of an active ingredient that elicits the desired biological or medicinal response in a subject's biological system without the risks outweighing the benefits of such response in accordance with the Federal Food, Drug, and Cosmetic Act, as amended (§§ 201-902, 52 Stat. 1040 et seq., as amended; 21 U.S.C. §§ 321-392). Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug. Efficacy is often measured by determining whether an active pharmaceutical ingredient demonstrates a health benefit over a placebo or other intervention when tested in an appropriate situation, such as a tightly controlled clinical trial.

The term “acceptable” with respect to a formulation, composition or ingredient, as used herein, means that the beneficial effects of that formulation, composition or ingredient on the general health of the male human being treated substantially outweigh its detrimental effects, to the extent any exist. In some embodiments, administration of a safe and effective amount of the anti-androgen results in no more than a grade 2 adverse event. In other embodiments, administration of a safe and effective amount of anti-androgen results in no more than a grade 3 adverse event. In other embodiments, administration of a safe and effective amount of anti-androgen results in no more than a grade 4 adverse event.

In some embodiments, the anti-androgen is present in a solid oral dosage form. In some embodiments, the anti-androgen is formulated as a tablet. In some embodiments, the anti-androgen is apalutamide. In some embodiments, the anti-androgen is enzalutamide. Solid oral dosage forms containing either apalutamide or enzalutamide may be provided as soft gel capsules as disclosed in WO2014113260 and CN104857157, each of which is incorporated herein by reference, or as tablets as disclosed in WO2016090098, WO2016090101, WO2016090105, and WO2014043208, each of which is incorporated herein by reference. Techniques suitable for preparing solid oral dosage forms of the present invention are described in Remington's Pharmaceutical Sciences, 18th edition, edited by A R. Gennaro, 1990, Chapter 89, and in Remington—The Science, and Practice of Pharmacy, 21st edition, 2005, Chapter 45. To prepare the pharmaceutical compositions of this invention, the active pharmaceutical ingredient is intimately admixed with a pharmaceutical carrier according to conventional pharmaceutical compounding techniques, which carrier may take a wide variety of forms depending on the form of preparation desired for administration (e.g., oral or parenteral). Certain suitable pharmaceutically acceptable carriers are known in the art. Descriptions of some of these pharmaceutically acceptable carriers may be found in The Handbook of Pharmaceutical Excipients, published by the American Pharmaceutical Association and the Pharmaceutical Society of Great Britain.

In solid oral preparations such as, for example, dry powders for reconstitution or inhalation, granules, capsules, caplets, gelcaps, pills and tablets (each including immediate release, timed release and/or sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

In certain embodyments these compositions are in unit dosage forms from such as tablets, pills, capsules, dry powders for reconstitution or inhalation, granules, lozenges, sterile solutions or suspensions, metered aerosol or liquid sprays, drops, or suppositories for administration by oral, intranasal, sublingual, intraocular, transdermal, rectal, vaginal, dry powder inhaler or other inhalation or insufflation means.

These formulations are manufactured by conventional formulation techniques. For preparing solid pharmaceutical compositions such as tablets, the principal active ingredient is mixed with a pharmaceutical carrier, e.g., conventional tableting ingredients such as diluents, binders, adhesives, disintegrants, lubricants, antiadherents, and glidants. Suitable diluents include, but are not limited to, starch (i.e. corn, wheat, or potato starch, which may be hydrolyzed), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e. AVICEL microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable glidants include, but are not limited to, talc, cornstarch, silica (i.e. CAB-O-SIL silica available from Cabot, SYLOID silica available from W.R. Grace/Davison, and AEROSIL silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Binders suitable for use in the pharmaceutical compositions provided herein include, but are not limited to, starches, cellulose, and its derivatives (e.g., ethylcellulose, cellulose acetate, carboxymethyl cellulose calcium, sodium carboxymethyl cellulose, methylcellulose, hydroxypropyl methylcellulose), polyvinyl pyrrolidone, and mixtures thereof.

Examples of fillers suitable for use in the pharmaceutical compositions provided herein include, but are not limited to, microcrystalline cellulose, powdered cellulose, mannitol, lactose, calcium phosphate, starch, pre gelatinized starch, and mixtures thereof.

The binder or filler in pharmaceutical compositions is typically present in from about 50 to about 99 weight percent of the pharmaceutical composition or dosage form.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in the pharmaceutical compositions provided herein include, but are not limited to, croscarmellose sodium, crospovidone, sodium starch glycolate, potato or tapioca starch, pre gelatinized starch, other starches, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, polyethylene glycol, other glycols, stearic acid, sodium lauryl sulfate, sodium stearyl fumarate, talc, hydrogenated vegetable oil (e.g., peanut oil, cottonseed oil, sunflower oil, sesame oil, olive oil, com oil, and soybean oil), zinc stearate, ethyl oleate, ethyl laureate, agar, and mixtures thereof. Lubricants are typically used in an amount of less than about 1 weight percent of the pharmaceutical compositions or dosage forms into which they are incorporated.

Compressed tablet formulations may optionally be film-coated to provide color, light protection, and/or taste-masking. Tablets may also be coated so as to modulate the onset, and/or rate of release in the gastrointestinal tract, so as to optimize or maximize the biological exposure of the patient to the API. Hard capsule formulations may be produced by filling a blend or granulation of apalutamide or enzalutamide into shells consisting of, for example, gelatin, or hypromellose.

Soft gel capsule formulations may be produced.

Pharmaceutical compositions intended for oral use may be prepared from the solid dispersion formulations, and blended materials described above in accordance with the methods described herein, and other methods known to the art for the manufacture of pharmaceutical compositions. Such compositions may further contain one or more agents selected from the group consisting of sweetening agents, flavoring agents, coloring agents, and preserving agents in order to provide pharmaceutically elegant and palatable preparations.

Tablets may contain the active ingredient in admixture with non-toxic pharmaceutically acceptable excipients that are suitable for the manufacture of tablets. These excipients may be for example, inert diluents, granulating, and disintegrating agents, binding agents, glidants, lubricating agents, and antioxidants, for example, propyl gallate, butylated hydroxyanisole, and butylated hydroxy toluene. The tablets may be uncoated or they may be film coated to modify their appearance or may be coated with a functional coat to delay disintegration, and absorption in the gastrointestinal tract, and thereby provide a sustained action over a longer period.

Compositions for oral use may also be presented as capsules (e.g., hard gelatin) wherein the active ingredient is mixed with an inert solid diluent, for example, calcium carbonate, calcium phosphate or starch, or as soft gelatin capsules wherein the active ingredient is mixed with liquids or semisolids, for example, peanut oil, liquid paraffin, fractionated glycerides, surfactants or olive oil. Aqueous suspensions contain the active materials in mixture with excipients suitable for the manufacture of aqueous suspensions. Dispersible powders and granules suitable for preparation of an aqueous suspension by the addition of water provide the active ingredient in mixture with a dispersing or wetting agent, suspending agent, and one or more preservatives. In certain embodiments of the invention, the pharmaceutical compositions of the invention include a diluent system, disintegrant, salt, lubricant, glidant, and film coat, at concentrations of from about 3% w/w to about 58% w/w, from about 4% w/w to about 20% w/w, from about 4% w/w to about 20% w/w, from about 0.5% w/w to about 4% w/w, from about 0% w/w to about 2% w/w, and from about 1% w/w to about 5% w/w respectively, or at from about 18% w/w to about 40% w/w, from about 7% w/w to about 15% w/w, from about 7% w/w to about 18% w/w, from about 1.0% w/w to about 3.0%, from about 0.1% w/w to about 1.0% w/w, and from about 2.0% w/w to about 4.0% w/w, respectively. In certain embodiments, the solid dispersion formulations are blended with a diluent, one or more disintegrating agents, lubricants, and glidants. An exemplary blended composition or oral dosage form includes mannitol, microcrystalline cellulose, croscarmellose sodium, sodium chloride, colloidal silica, sodium stearyl fumarate, and magnesium stearate.

The disintegrant may be present in a concentration from about 4% w/w to about 20% w/w or from about 7% w/w to about 15% w/w. A salt may be also present, which may be sodium chloride, potassium chloride or a combination thereof. The combination of salts and disintegrant is present at a concentration from about 5% w/w to about 35% w/w of the final pharmaceutical composition.

In certain embodiments, inactive ingredients of the core tablet may include: colloidal anhydrous silica, croscarmellose sodium, hydroxypropyl methylcellulose-acetate succinate, magnesium stearate, microcrystalline cellulose, and silicified microcrystalline cellulose. In other embodiments, the tablets are finished with a film-coating consisting of the following excipients: iron oxide black, iron oxide yellow, polyethylene glycol, polyvinyl alcohol, talc, and titanium dioxide

In other embodiments, a single unit dosage of the pharmaceutical composition of the present disclosure may comprise, consist of, or consist essentially of about 300 mg of darolutamide. In some embodiments, multiple doses of the single unit dosage pharmaceutical composition comprising, consisting of, or consisting essentially of about 300 mg of enzalutamide, e.g., 2 multiple or individual unit dosage forms, are administered to the human. The total daily dose of darolutamide may be in the range of about 300 mg per day to about 1500 mg per day. In certain embodiments, the total daily dose of darolutamide may be about 1200 mg per day. The darolutamide compound may exists as any of the possible diasetereoisomers including mixtures of any combination of the diastereoisomers. In certain embodiments, the darolutamide may have a structure in the form of one or more of the (R,R) diastereomer, the (R,S) diastereoisomer, the (S,R) diastereoisomer, and the (S,S) diastereoisomer, including various mixtures of two or more of the possible diastereoisomers in any ratio. In certain embodiments, the darolutamide may have a structure in the form of the (S,R) diastereoisomer, the (S,S) diastereoisomer and any isomeric ratio of the (S,S) diastereomer and the (S,R) diastereomer.

In some embodiments, a single unit dosage of the pharmaceutical composition comprises, consists of, or consists essentially of about 60 mg of apalutamide. In some embodiments, multiple doses of the single unit dosage pharmaceutical composition comprising, consisting of, or consisting essentially of about 60 mg of apalutamide, e.g., 4 multiple or individual unit dosage forms, are administered to the human. The total daily dose of apalutamide may be about 240 mg per day.

In further embodiments, a single unit dosage of the pharmaceutical composition comprises, consists of, or consists essentially of about 40 mg of enzalutamide. In some embodiments, multiple doses of the single unit dosage pharmaceutical composition comprising, consisting of, or consisting essentially of about 40 mg of enzalutamide, e.g., 4 multiple or individual unit dosage forms, are administered to the human. The total daily dose of enzalutamide may be about 160 mg per day.

In further embodiments, a single unit dosage of the pharmaceutical composition comprises, consists of, or consists essentially of at least one anti-androgen, such as at least one androgen receptor inhibitor. According to certain embodiments, the at least one anti-androgen may be at least one androgen receptor inhibitor selected from the group consisting of darolutamide, apalutamide, enzalutamide, and combinations thereof. In some embodiments, multiple doses of the single unit dosage pharmaceutical composition comprising, consisting of, or consisting essentially of from about 40 mg to 300 mg of the androgen receptor inhibitor, e.g., 2 to 4 multiple or individual unit dosage forms, are administered to the human. The total daily dose of the anti-androgen may range from about 160 mg per day to about 1500 mg per day.

All formulations for oral administration are in dosage form suitable for such administration.

### Methods Of Dosing and Treatment Regimens

In one aspect, described herein are methods of treating BCR hormone sensitive prostate cancer comprising, consisting of, or consisting essentially of administering a safe and effective amount of an anti-androgen to a male human with a BCR hormone sensitive prostate cancer. In certain embodiments, the anti-androgen compound may be an androgen receptor inhibitor, such as an androgen receptor inhibitor selected from the group consisting of darolutamide, apalutamide, enzalutamide, and combinations thereof. The anti-androgen composition or formulation may be administered orally. In some embodiments, the anti-androgen is administered daily. In some embodiments, the anti-androgen is administered twice-a-day. In some embodiments, the anti-androgen is administered three times a day. In some embodiments, the anti-androgen is administered four times a day. In certain embodiments, the doses of anti-androgen compound employed for treatment of the diseases or conditions described herein in humans are typically in the range of about 10 mg per day to about 1500 mg per day.

In certain embodiments, the anti-androgen compound may be darolutamide. According to embodiments, the darolutamide is administered orally. In some embodiments, the anti-androgen is administered daily. In some embodiments, the anti-androgen is administered twice-a-day. In some embodiments, the anti-androgen is administered three times a day. In some embodiments, the anti-androgen is administered four times a day. In some embodiments, the apalutamide is administered every other day. In some embodiments, the anti-androgen is administered weekly. In some embodiments, the anti-androgen is administered twice a week. In some embodiments, the anti-androgen is administered every other week. In some embodiments, the anti-androgen is administered orally on a continuous daily dosage schedule.

In one embodiment, the desired dose is conveniently presented in a single dose or in divided doses administered simultaneously (or over a short period of time) or at appropriate intervals, for example as two, three, four or more sub-doses per day. In some embodiments, the anti-androgen is conveniently presented in divided doses that are administered simultaneously (or over a short period of time) once a day. In some embodiments, the anti-androgen is conveniently presented in divided doses that are administered in equal portions twice-a-day. In some embodiments, the anti-androgen is conveniently presented in divided doses that are administered in equal portions three times a day. In some embodiments, the anti-androgen is conveniently presented in divided doses that are administered in equal portions four times a day.

In some embodiments, the anti-androgen is a second-generation anti-androgen, for example selected from the group consisting of darolutamide, enzalutamide, apalutamide and mixtures of any thereof. In certain embodiments, the anti-androgen is darolutamide. In some embodiments, the anti-androgen is enzalutamide. In some embodiments, the anti-androgen is apalutamide.

In general, doses of darolutamide employed for treatment of the diseases or conditions described herein in humans are typically in the range of about 300 mg per day to about 1500 mg per day.

In some embodiments, darolutamide is administered orally at a dose of about 1200 mg per day. In some embodiments, greater than 1200 mg per day of darolutamide is administered. In some embodiments darolutamide is administered orally to the human at a dose of about 600 mg twice daily.

In some embodiments, enzalutamide is administered orally at a dose of about 160 mg per day. In some embodiments, greater than 160 mg per day of enzalutamide is administered. In general, doses of apalutamide employed for treatment of the diseases or conditions described herein in humans are typically in the range of 10 mg to 1000 mg per day. In some embodiments, apalutamide is administered orally to the human at a dose of about 30 mg per day to about 600 mg per day. In some embodiments, apalutamide is administered orally to the human at a dose of about 30 mg per day, about 60 mg per day, about 90 mg per day, about 120 mg per day, about 160 mg per day, about 180 mg per day, about 240 mg per day, about 300 mg per day, about 390 mg per day, about 480 mg per day, about 600 mg per day, about 780 mg per day, about 960 mg per day, or about 1200 mg per day.

In some embodiments, apalutamide is administered orally to the human at a dose of about 240 mg per day. In some embodiments, greater than 240 mg per day of apalutamide is administered to the human. In some embodiments, apalutamide is administered orally to the human at a dose of about 60 mg four times per day. In some embodiments, apalutamide is administered orally to the human on a continuous daily dosing schedule.

In certain embodiments wherein improvement in the status of the disease or condition in the human is not observed, the daily dose of anti-androgen is increased. In some embodiments, a once-a-day dosing schedule is changed to a twice-a-day dosing schedule. In some embodiments, a three times a day dosing schedule is employed to increase the amount of anti-androgen that is administered.

In some embodiments, the amount of anti-androgen that is given to the human varies depending upon factors such as, but not limited to, condition and severity of the disease or condition, and the identity (e.g., weight) of the human, and the particular additional therapeutic agents that are administered (if applicable).

### Kits and Articles of Manufacture

For use in the methods of use described herein, kits and articles of manufacture are also described. Such kits include a package or container that is compartmentalized to receive one or more dosages of the pharmaceutical compositions disclosed herein. Suitable containers include, for example, bottles. In one embodiment, the containers are formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252.

Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert.

In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1—Pre-Clinical Development

Darolutamide (BAY 1841788, ODM-201) is a second-generation anti-androgen that binds directly to the ligand-binding domain of the AR, impairing nuclear translocation, AR binding to DNA and inhibiting AR target gene and protein levels, thereby inhibiting tumor growth (A. Moilanen et al., Sci. Rep., 2016, 5:12007; T. Sugawara et al., Int. J. Cancer, 2019, 14:1382-1394; S. J. Baumgart, Mol. Oncol., 2021, 14:2022-2039). Darolutamide binds to the AR with greater affinity than bicalutamide and inhibits prostate tumor progression, as shown in several cell line-and patient derived models (A. Moilanen et al., Sci. Rep., 2016, 5:12007; T. Sugawara et al., Int. J. Cancer, 2019, 14:1382-1394). It consists of two diastereomers, ORM-16497 and ORM-16555, with comparable activity (T. Sugawara et al., Int. J. Cancer, 2019, 14:1394). It has activity against known AR mutants that confer resistance to other second-generation antiandrogens (H. Borgmann et al., 2018, Eur. Urol., 2018, 73:4-8; T. Sugawara et al., Int. J. Cancer, 2019, 14:1394).

Darolutamide shows high bioavailability with complete and rapid absorption in rats (P. Taavitsainen et al., Xenobiotica, 2020, 50:967-979). Rapid interconversion of the diastereoisomers (S,R)- and (S,S)-via the major circulating metabolite keto-darolutamide is observed both in vitro and in vivo. The (S,R) diastereoisomer has faster elimination although the diastereoisomer ratio remains unchanged at high doses. In vivo distribution studies performed with [¹⁴C]-darolutamide show wide and homogenous distribution in all tissues except the brain, following single oral administration (P. Taavitsainen et al., Xenobiotica, 2020, 50:967-979). In contrast, exposure in prostate tissue was higher, demonstrating effective tissue penetration at the target organ for therapeutic activity (P. Taavitsainen et al., Xenobiotica, 2020, 50:967-979).

In vitro, darolutamide is mainly metabolized via oxidative biotransformation catalyzed by Cytochrome P450 (CYP)3A4. Only one major metabolite, keto-darolutamide, was observed in plasma of all investigated species including man (P. Taavitsainen et al., 2021 Drug Metab Dispos 49:420-433. It is a substrate for P-glycoprotein (P-gp) and breast cancer resistance protein (BCRP) (C. Zurth et al., Eur. J. Drug Metab. Pharmacokinet., 2019, 44:747-759). The enzymatic activity of nine CYP isoforms was not inhibited or slightly inhibited in vitro with darolutamide, and a rank order and mechanistic static assessment indicated that risk of clinically relevant drug-drug interactions (DDIs) via CYP inhibition is very low. In vitro, darolutamide exhibited no relevant induction of CYP1A2 or CYP2B6 activity. Inhibition of BCRP-, P-gp-, organic anion transporter (OAT)3-, multidrug and toxin extrusion (MATE)1-, MATE2-K-, organic anion-transporting polypeptide (OATP)1B1- and OATP1B3-mediated transport was observed in vitro. Phase I data showed that darolutamide exposure increased 1.75-fold with co-administered itraconazole and decreased by 72% with rifampicin. Co-administration of darolutamide with CYP3A4/P-gp substrates showed no effect or only minor effects. Rosuvastatin exposure increased 5.2-fold with darolutamide because of BCRP and probably also OATPB1/OATPB3 inhibition. In conclusion, Darolutamide has a low potential for clinically relevant DDIs with drugs that are substrates for CYP or P-gp; increased exposure of BCRP and probably OATP substrates was the main interaction of note (C. Zurth et al., Eur. J. Drug Metab. Pharmacokinet., 2019, 44:747-759).

The safety profile of darolutamide was characterised in rats and dogs through the conduct of repeat-dose toxicity studies of up to 6- and 9-month, toxicokinetic data, genotoxicity and phototoxicity studies. Consistent with the pharmacologic activity, the primary target organ was the reproductive system. In repeated dose toxicity studies in rats and dogs, the main findings were changes in the male reproductive organs (decreases in organ weight with atrophy of the prostate and epididymides). These effects occurred at systemic exposures in the range of or below the anticipated human exposure (based on AUC comparison). Additional changes to reproductive tissues included minimal increase in vacuolation of the pituitary gland, atrophy and secretory reduction in seminal vesicles and mammary glands in rats as well as testicular hypospermia, seminiferous tubule dilatation and degeneration in dogs. Changes in the male reproductive organs in both species were consistent with the pharmacological activity of darolutamide and reversed or partially resolved after 4- to 8-week recovery periods. No other adverse effects were observed. No deaths occurred during the treatment period.

Darolutamide is considered as a non-phototoxic and non-genotoxic compound based on available data (nubeqa-epar-public-assessment-report_en_final_Apr2020).

In a transgenic mouse study in male and female 001178-T (hemizygous) RasH 2 mice administered up to 500 mg/kg/dose BID darolutamide for at least 26 weeks no carcinogenic potential for darolutamide was observed.

### Example 2—Clinical Development

A randomized, double-blind, placebo-controlled Phase 3 study of darolutamide plus androgen deprivation therapy (ADT) compared with placebo plus ADT in patients with high-risk biochemical recurrence (BCR) of prostate cancer

### Primary Objective

To determine if darolutamide plus ADT given for 24 months improves rPFS by PSMA PET/CT compared with placebo plus ADT given for 24 months

### Secondary Objectives

To further evaluate efficacy and to measure the treatment impact on patients' quality of life comparing the following parameters in men with BCR of hormone sensitive prostate cancer treated with darolutamide versus placebo: MFS by CI assessed by BICR, Time to CRPC assessed by investigator, time to initiation of first subsequent systemic antineoplastic therapy, time to loco-regional progression by PSMA PET/CT, time to first SSE, OS, PSA undetectable rates (<0.2 ng/ml) at 12 months, time to deterioration in the FACT-P total

Score and safety and tolerability.

### Other Objectives

To further evaluate efficacy and to measure the treatment impact on patient's quality of life comparing; time to PSA progression PSA undetectable rates (<0.2 ng/ml) at any time, time to testosterone recovery, time to worsening in prostate cancer by EORTC QLQ-PR25 subscales and total score, time to deterioration in the PCS subscale of FACT-P.

To evaluate biomarkers to investigate the pharmacodynamic effects of darolutamide and molecular subgroups with distinct prognosis and/or response to darolutamide

To further investigate the study drug and similar drugs (e.g., mode-of-action-related effects, safety) and to further investigate patho-mechanisms deemed relevant to cancer and associated health problems.

### Study Design

This is a randomized, double-blind, placebo-controlled Phase 3 study to evaluate if the addition of darolutamide to ADT given for 24 months provides superior efficacy compared with 24 months of placebo plus ADT based on rPFS by PSMA PET/CT as assessed by BICR in participants with hormone-sensitive high-risk BCR of prostate cancer.

The primary endpoint rPFS by PSMA PET/CT assessed by BICR is defined as the time from randomization to first documentation of at least one new distant metastatic lesion by PSMAPET/CT assessed by BICR or death due to any cause, whichever occurs first.

The secondary endpoint MFS by conventional imaging assessed by BICR is defined as the time from randomization to first documentation of at least one new distant metastatic lesion by CI assessed by BICR or death due to any cause, whichever occurs first

Approximately 750 study participants will be randomly assigned in a 1:1 ratio to receive one of the following study drugs for 24 months.
Darolutamide 600 mg (2 tablets of 300 mg) twice daily (BID) with food, equivalent to a total daily dose of 1200 mg
   OR
Placebo darolutamide matched tablets in appearance, BID with food

Background therapy: All participants must receive ADT (LHRH agonist/antagonists) of the investigator's choice as standard therapy for 24 months.

Image-guided radiotherapy or surgery of distant and/or loco-regional lesions identified by PSMA PET/CT will be allowed for all participants.

Participants will be stratified at randomization (Section 6.9) as follows:
PSADT <6 months vs. ≥6 to <12 months
Prior radical prostatectomy vs. primary radiotherapy
Distant metastasis (with or without locoregional lesions) vs. locoregional lesions only

The study will comprise 4 consecutive periods: Screening, Treatment, Active follow-up, and Long-term (with clinical visits and Survival) follow-up. Screening must occur within 30 days prior to randomization. The start of the Treatment period is defined by the first administration of study treatment, which has to occur within 3 days after date of randomization and after all Screening period activities. Participants will receive study treatment for 24 months unless, during the 24 months Treatment period, there is disease progression, unacceptable toxicity.

The screening visit needs to be recorded in the Interactive Response Technology (IRT) system, where the participant will be assigned a unique identification number (ID number).

Some Screening and Active follow-up visit procedures may be conducted using eTools where applicable except for PSMA PET/CT, CT/MRI, and bone scans.

In addition to central laboratory sample collection, a local PSA value should be obtained at screening and is used to confirm the patient has PSADT <12 months the last 12 months before randomization, at least 4 weeks apart. PSADT should be obtained when patient was under normal values of testosterone or with testosterone blood levels recovered to >150 ng/ml after discontinuation of neoadjuvant and/or adjuvant therapy.

PSA values used in calculation of PSADT should preferably have been obtained using the same assay and if possible, at the same laboratory. Screening PSA from local laboratory must be included in the PSADT calculation. PSA values used for the calculation of PSADT must be >0.2 ng/ml and should follow a global rising trend. All included PSA values should be obtained within the past 12 months at most, to reflect the current disease activity [AUA/ASTRO/SUO Guidelines 2020 and EAU-EANM-ESTRO-ESUR-ISUP-SIOG Guidelines 2022, (Lowrance et al. 2021, Mottet etal. 2022)]. PSA values used for the calculation of PSADT will be recorded in the CRF. PSADT will be calculated according to AUA/ASTRO/SUO Guidelines 2020 and EAU-EANM-ESTRO-ESUR-ISUP-SIOG Guidelines 2022.

Patients with PSMA PET/CT positive for tumor lesions which are also detectable on screening CI (bone scan and CT/MRI) do not meet eligibility criteria and should not be re-screened as they can be treated with standard of care (SoC) available for mHSPC or loco-regional therapy according to local clinical practice.

### Study Population

Male ≥18 years of age at the time of signing the informed consent.

### Inclusion Criteria

Subjects enrolled in this study are required to meet the following key acceptance criteria:
Capable of giving signed informed consent.
Histologically or cytologically confirmed adenocarcinoma of prostate.
Prostate cancer initially treated by:
   radical prostatectomy (RP) followed by adjuvant radiotherapy (ART) or salvage radiotherapy (SRT),
   or RP in participants who are unfit for ART or SRT,
   or primary radiotherapy (RT)
High-risk biochemical recurrence (BCR), defined as prostate-specific antigen doubling time (PSADT)<12 months
AND
   PSA≥0.2 ng/ml after ART or SRT post RP or after RP in participants who are unfit for ART or SRT, or
   PSA≥2 ng/ml above the nadir after primary RT only
Participants must undergo prostate-specific membrane antigen positron emission tomography/computed tomography (PSMA PET/CT) within the 30-day Screening period using either 18F-DCFPyL (piflufolastat F 18) or 68Ga-PSMA-11 which will be assessed by BICR to identify
   at least one PSMA PET positive lesion of prostate cancer
   AND
   the number and the location of lesions to be used as baseline reference.
Serum testosterone ≥150 ng/dl (5.2 nmol/L).
Eastern Cooperative Oncology Group (ECOG) performance status of 0 or 1.
Blood counts at screening:
   Hemoglobin ≥9.0 g/dL (participant must not have received blood transfusion within 7 days prior to sample being taken)
   Absolute neutrophil count (ANC) ≥1.5×109/L (participant must not have receive any growth factor within 4 weeks prior to sample being taken)
   Platelet count ≥100×109/L
Screening values of:
   Alanine aminotransferase (ALT)≤1.5×upper limit of normal (ULN)
   Aspartate aminotransferase (AST)≤1.5×ULN
   Total bilirubin (TBL)≤1.5 ULN, (except participants with a diagnosis of Gilbert's disease)
   Estimated glomerular filtration rate (eGFR) >40 ml/min/1.73 m2 calculated by the CKD-EPI formula
Sexually active male participants must agree to use contraception as detailed in the protocol during the Treatment period and for at least 3 months after the last dose of study treatment, and refrain from donating sperm during this period.

### Exclusion Criteria

Participants are excluded from the study if any of the following criteria apply:
Pathological finding consistent with small cell, ductal or ≥50% component of neuroendocrine carcinoma of the prostate.
History of bilateral orchiectomy.
Metastases or recurrent/new malignant lesions in prostate gland/bed seminal vesicles, lymph nodes below the CIA bifurcation on conventional imaging (CI) as assessed by BICR during screening.
Brain metastasis on PSMA PET/CT by BICR at screening.
High-risk BCR after primary radiotherapy with new loco-regional lesions on screening PSMA PET/CT who are eligible for curative salvage prostatectomy.
   Note: Participants treated with curative salvage prostatectomy after primary RT who meet the PSA criteria (inclusion criteria 5) may be considered for the study.
Prior treatment with second generation (e.g. enzalutamide, apalutamide) androgen receptor inhibitors (ARIs) and CYP 17 inhibitors (e.g., abiraterone) within 18 months prior to signing of the ICF.
Prior treatments with PSMA-radiotherapeutics within 12 months prior to randomization.
Prior radiotherapy (including image-guided radiotherapy) as primary, adjuvant or salvage treatment completed within 8 weeks prior to signing of the ICF.
Known hypersensitivity to any of the study drugs, imaging agents, study drug classes, or excipients in the formulation of the study drugs.
Contraindication to PSMA PET/CT tracer, or both CT and MRI contrast agents
Any prior malignancy (other than adequately treated basal cell or squamous cell skin cancer, superficial bladder cancer, or any other cancer in situ currently in complete remission) within 5 years.
History of pelvic radiotherapy for other malignancy.
Ongoing or active infection (bacterial, fungal, or viral including Hepatitis viral and Hepatitis B reactivation) requiring systemic therapy.
Any positive test result for Hepatitis B virus (HBV) or Hepatitis C virus (HCV) indicating the presence of virus.
   Active HBV (chronic or acute; defined as having a known positive Hepatitis B
   surface antigen [HBsAg] test at the time of screening) except for participants on
   antiviral therapy for HBV with an undetectable or low viral load
   Participants with past HBV infection or resolved HBV infection (defined as the presence of Hepatitis B core antibody [HbcAb] and absence of HbsAg) are eligible if HBV DNA is negative
   Participants positive for HCV antibody unless polymerase chain reaction is negative for HCV RNA
   Note: Hepatitis B and C testing is not required unless mandated by local authority.
Known human immunodeficiency virus (HIV) infection with any of the following:
   CD4+ T-cell (CD4+) count of less than 350 cells/μL
   History of AIDS defining opportunistic infection within the past 12 months
   On established antiretroviral therapy for less than 4 weeks
   Presenting with a viral load of more than 400 copies/mL prior to enrollment
   On antiretroviral therapy or prophylactic antimicrobials that are expected to cause significant drug-drug interactions or overlapping toxicities with study treatment and cannot be changed to alternative agents.
   Note: HIV testing is not required unless mandated by local authority.
Had any of the following within 6 months before randomization: stroke, myocardial infarction, severe/unstable angina pectoris, coronary/peripheral artery bypass graft, congestive heart failure (New York Heart Association Class III or IV).
Hypertension as indicated by a resting systolic blood pressure (BP) >140 mmHg or diastolic BP ≥100 mmHg despite medical management.

A gastrointestinal disorder or procedure which is expected to interfere significantly with absorption of study drug.
Previous (within 28 days before the start of darolutamide/placebo or 5 half-lives of the investigational treatment of the previous study, whichever is longer) or concomitant participation in another clinical study with investigational medicinal product(s).
Any other serious or unstable illness, or medical, social, or psychological condition, that could jeopardize the safety of the participant and/or his/her compliance with study procedures or may interfere with the participant's participation in the study or evaluation of the study results.
Inability to swallow oral medications

### Discontinuation/Withdrawal Criteria

All participants who enter the study should complete all applicable study periods. Participants can be withdrawn from any study period at any time. Discontinuation of study treatment alone does not constitute withdrawal from the study.

### Discontinuation of Study Treatment

In this study treatment is administered for a duration of 24 months.

After completion of 24 months of study treatment, participants must discontinue study treatment and will enter Active or Long-term follow-up. In some instances, it may be necessary for a participant to permanently discontinue (definitive discontinuation) study treatment before completing the planned 24 months of treatment. After the study treatment is definitively discontinued, the participant will remain in the study to be evaluated for primary, secondary, and other pre-specified endpoints.

Participants must be withdrawn from the study drug if any of the following occurs:
If, in the investigator's opinion, continuation of the study drug would be harmful to the participant's well-being. If a participant experiences a clinical progression by worsening of disease signs/symptoms leading to study treatment discontinuation, radiological evaluations must be continued until progression by PSMA PET/CT established by BICR or systemic antineoplastic therapy is started
Disease progression (rPFS by PSMA PET-CT established by BICR)
Start of new antineoplastic therapy
Unacceptable toxicity
Study drug interruption >28 consecutive days
Darolutamide dosing below 300 mg BID
Occurrence of Grade 23 study drug-related TEAE while the participant is on 300 mg BID
Hepatic transaminase elevations suggestive of idiosyncratic drug-induced liver injury (DILI) considered to be causally related to study drug
Development of any intercurrent illness or situation which may, in the judgment of the investigator, affect assessment of clinical status and study endpoints to a relevant degree
Development of a second primary malignancy that requires a different treatment

1. A method of improving progression-free survival in a male human with a biochemical recurrence of prostate cancer, said method comprising administering to said male human a drug product comprising an androgen receptor inhibitor.

2. The method of claim 1 wherein the progression-free survival is radiological progression-free survival by prostate-specific membrane antigen positron emission tomography.

3. The method of claim 1 wherein the progression free survival is radiological progression-free survival by computed tomography.

4. The method of claim 1 wherein the biochemical recurrence of prostate cancer is a high-risk biochemical recurrence of prostate cancer.

5. The method of claim 1 wherein the androgen receptor inhibitor is darolutamide.

6. The method of claim 1 further comprising administering an androgen deprivation therapy.

7. The method of claim 6 wherein administering the androgen deprivation therapy is performed sequentially or concomitantly to the administering the drug product comprising the androgen receptor inhibitor.

8. The method of claim 6 wherein the androgen deprivation therapy comprising of orchiectomy or LHRH agonists or LHRH antagonists.

9. A method of improving radiological progression-free survival by prostate-specific membrane antigen positron emission tomography or computed tomography in a male human with a high-risk biochemical recurrence of prostate cancer, said method comprising administering to said male human a drug product comprising an androgen receptor inhibitor wherein the androgen receptor inhibitor comprises darolutamide in sequential or concomitant combination with androgen deprivation therapy comprising of orchiectomy or administering LHRH agonists or antagonists.

10. A method of treating a biochemical recurrence of prostate cancer in a human male, the method comprising administering a drug product comprising an anti-androgen to the human male.

11. The method of claim 10, wherein the anti-androgen is an androgen receptor inhibitor.

12. The method of claim 11, wherein the androgen receptor inhibitor is a second-generation androgen receptor inhibitor selected from the group consisting of darolutamide, enzalutamide, apalutamide, and combinations of any thereof.

13. The method of claim 12, wherein the second-generation androgen receptor inhibitor is administered in a daily dose ranging from about 10 mg per day to about 1500 mg per day

14. The method of claim 10, wherein the androgen receptor inhibitor is darolutamide.

15. The method of claim 14, wherein the darolutamide is administered in a daily dose ranging from about 300 mg per day to about 1500 mg per day.

16. The method of claim 14, wherein the darolutamide is administered in a daily dose of about 1200 mg per day.

17. The method of claim 14, wherein the darolutamide is in the form of the (S,S) diastereomer, the (S,R) diastereomer, and any isomeric ratio of the (S,S) diastereomer and the (S,R) diastereomer.

18. The method of claim 10, further comprising administering an androgen deprivation therapy.

19. The method of claim 17, wherein administering the androgen deprivation therapy is performed sequentially or concomitantly to the administering the drug product.

20. Darolutamide in combination with LHRH agonists or LHRH antagonists for improving radiological progression-free survival by prostate-specific membrane antigen positron emission tomography or computed tomography in a male human with a high-risk biochemical recurrence of prostate cancer.
